(19) United States
(12) Patent Application Publication
(10) Pub. No.: US 2008/0208145 A1
(43) Pub. Date: Aug. 28, 2008

(54) DISPOSABLE SHOWER GUARD FOR RENAL ACCESS CATHETER

(76) Inventor: James S. McCulloch, St. Peters, MO (US)

Correspondence Address: HUSCH BLACKWELL SANDERS LLP, 720 OLIVE STREET, SUITE 2400, ST. LOUIS, MO, 63101, US

(21) Appl. No.: 11/678,720

(22) Filed: Feb. 26, 2007

(51) Int. Cl. A61M 5/32

(52) U.S. Cl. 604/263

(57) ABSTRACT

A hemodialysis catheter, also know as a renal dialysis access catheter penetrates a patients skin at an entrance site which is protected by associated bandaging, all of which needs to be protected from water when the patient takes a shower. The single use, disposable shower guard surrounds and encloses the catheter, the entrance site and the associated protective bandaging. A generally rectangular cutout in the guard is sized and oriented to surround the protective bandaging and leave a margin between the edge of the cutout and the protective bandaging so that the bandaging will not be disturbed when the guard is removed.

### BACKGROUND OF THE INVENTION

The present invention is a disposable, single use guard that may be used by renal dialysis patients to protect the access catheter, the entrance site and associated protective bandaging from water during a shower.

Because of the risk of infection, all renal dialysis patients with access catheters are instructed not to take a shower. The inability to take a shower may disrupt the lifestyle of the patient. There is a recognized need in the field to develop some device to allow renal dialysis patients to return to a more normal lifestyle.

For example, U.S. Pat. No. 5,18,274 is entitled “Catheter Shower Shield” and is intended to protect a chemotherapy catheter and the entrance site from water while the patient takes a shower. This patented apparatus is like a large removable bib and is intended to be reused each time the patient showers. Unlike this prior art, the present invention is a disposable single use sleeve that encloses and surrounds the catheter and the entrance site. A similar reusable shower shield is disclosed in U.S. Pat. No. 6,571,395 entitled “Protective Guard for Catheter Exit Site” and is intended for use in connection with a hemodialysis catheter. This prior art is a waterproof drape that is intended to be reused each time the patient showers.

European Patent Application publication number 6 671 182 is entitled “Catheter Protector” and is intended to protect a catheter from water while showering. Many of the embodiments disclosed in the publication include a zip-like closing means allowing access to the catheter while the protector is in place on the patient. To the contrary, the present invention is a closed system to protect the catheter, the entrance site and any associated protective bandaging from water and water vapor.

Those familiar with renal dialysis will know that protection of the entrance site is important to the patient and medical personnel. Typically, medical personnel place a protective bandage over the entrance site and around the catheter after dialysis is complete. The protective bandage over the entrance site is generally rectangular in shape and aligned perpendicular to the longitudinal axis of the catheter. The present invention uses a generally rectangular cutout to surround the protective bandage over the entrance site with a margin between the edge of the cutout and the protective bandage. This leaves the associated protective bandage undisturbed during application of the disposable, single use guard of the present invention.

The aforementioned European Patent Application discloses a circular cutout to surround the entrance site as well as a rounded rectangular shape shown in FIGS. 5, 6 and 7. However, FIGS. 5, 6 and 7 show a peritoneal dialysis catheter and the disclosure does not mention proper orientation of the cutout or any associated protective bandage over the entrance site. The proper orientation of the cutout relative to the protective bandage over the entrance site and shape of the cutout of the present invention is not shown in this European Patent Application. The proper orientation and shape of the cutout of the present invention and is important to avoid disturbances to the associated protective bandage over the entrance site of a hemodialysis catheter.

Other publications disclose devices to protect central venous catheters, such as US 2005/0033241 and US 2002/0166559.

A variety of ostomy products use bags to collect fecal material. Many of these ostomy products use a circular cutout that is applied to a stoma, such as those disclosed in U.S. Pat. Nos. 5,730,736; 5,834,009; 6,206,864; 6,887,222; U.S. Patent Application Publications Nos. US 2003/2260786; US 2003/0153882; US 2005/0113770; US 2005/0131360; US 2006/0276763. Other products can be used for irrigating and draining of wounds, such as those disclosed in U.S. Pat. No. 5,236,426; U.S. Pat. No. 4,553,967 and Reissue 29,319.

### SUMMARY OF THE INVENTION

A disposable, single use shower guard for a renal dialysis access catheter requires proper orientation of a generally rectangular cutout over the generally rectangular protective bandage typically placed over the entrance site for the catheter. This is also sometimes referred to as a hemodialysis catheter. The guard includes a waterproof sleeve to receive the external portion of the catheter and surround the protective bandaging at the catheter entrance site. A wafer is attached to the waterproof sleeve and has adhesive placed on the side opposite the sleeve. The wafer and the adhesive is placed on the skin of the patient proximate the catheter entrance site. The generally rectangular cutout is sized to allow a margin between the edge of the cutout and the edge of the generally rectangular protective bandage over the entrance site for the catheter. One or more packages of desiccant may be positioned inside the guard to absorb water vapor. A colored outline may be placed around the edge of the cutout to facilitate positioning relative to the protective bandage.

When the patient wants to take a shower, the guard is placed around the external portion of the catheter and protective bandaging and the wafer is removably affixed to the patient's skin by an adhesive. The patient then takes a shower and afterwards dries off. The guard should then be removed from the patient after they have dried off.

### BRIEF DESCRIPTION OF DRAWINGS

FIG. 1 is an elevation view of a portion of the human torso with a renal dialysis catheter installed in the upper left chest without any protective bandaging.

FIG. 2 is an elevation view of the renal dialysis catheter of FIG. 1 with protective bandaging in place.

FIG. 3 is a section view along the line 3-3 of FIG. 2 of the renal dialysis catheter with protective bandaging in place.

FIG. 4 is a top view of the shower guard.

FIG. 5 is a rear view of the shower guard.

FIG. 6 is a section view of the shower guard along the line 6-6 of FIG. 4.

FIG. 7 is a section view of the shower guard surrounding a renal dialysis catheter that has been installed in a patent.

FIG. 8 is a top cut-a-way view of a portion of the shower guard and the renal dialysis catheter of FIG. 7.

### DETAILED DESCRIPTION

Referring to FIGS. 1-3, renal access catheter 20 is installed in the patient 22 in the upper left anterior quadrant of the chest 24. The catheter 20 passes from the exterior 26 of the patient to the chest cavity 28 of the patient at the catheter entrance site 30. A stitch 32 is typically placed in the patient's skin 34 and the renal access catheter to help hold the catheter in place.

The renal access catheter 20 includes an inlet tube 36, an inlet tube clamp 38, an inlet port 40 and a removable inlet port plug 42. The renal access catheter 20 further includes an outlet tube 44, an outlet tube clamp 46, an outlet port and a removable outlet port plug 48. The renal access catheter 20 is connected to a hemodialysis machine in a manner well known to those skilled in the art to purify the patient's blood due to total or partial kidney failure.

After the hemodialysis is complete, it is common to fold a 2′×2″ gauze bandage in half and place it over the catheter entrance site 30 and secure the upper protective bondage 60 with tape 62 which is generally clear. The purpose of this protective bandage is to help reduce the risk of infection at the catheter entrance site 30. After hemodialysis it is also common to wrap a 6′6″ gauze around the inlet and outlet tubes 36 and 34 and secure the lower protective bandage 64 with one or more pieces of tape 66 and 68. After placement of the upper protective 60 and the lower protective bandage 64, the patient may leave the hemodialysis clinic until it is time for the next treatment. However, in the interim between treatments, patients are cautioned not to shower or otherwise get the bandaging and the protective catheter entrance site wet. There is a need to develop an economical and reliable shower guard that can be easily put in place and removed by most patients and/or caregivers.

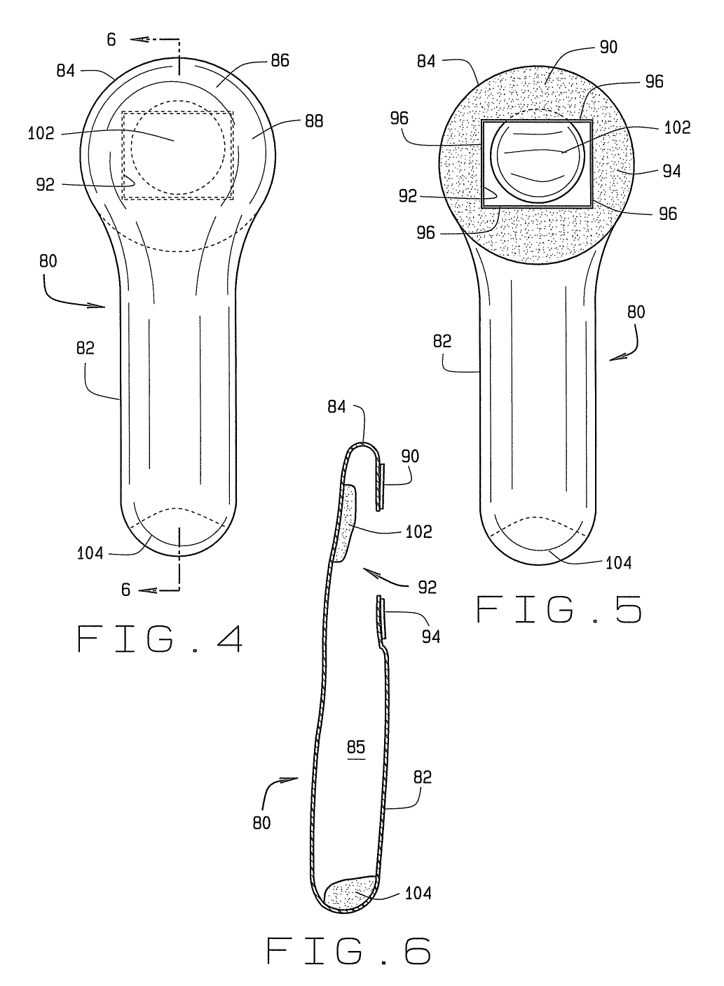

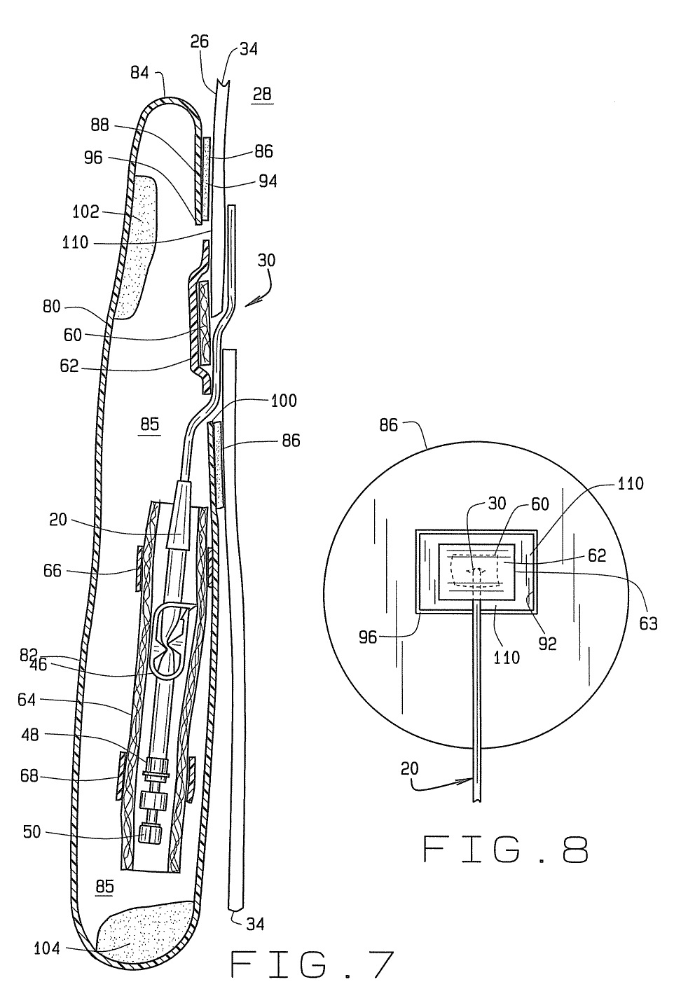

Referring now to FIGS. 4-6, the shower guard 80 is economical, reliable and easy to use by patients and/or caregivers, while the patient takes a shower. The shower guard 80 includes a sleeve 82 which depends from the cap 84. The shower guard 80 defines an interior chamber 85 which surrounds and protects the renal access catheter 20, as better seen in FIG. 7.

A two sided wafer 86 has an anterior side 88 connected to the shower guard 80 and a posterior side 90. The posterior side 90. The posterior side 90 of the wafer is coated with adhesive 94 to adhere to the patient's skin 38. A generally rectangular cutout 92 is formed in the two sided wafer. The cutout 92 is sized and arranged to fit over the upper protective bandage 60 and tape 62 as better seen in FIGS. 7 and 8. A colored edge 96 may be formed around the cutout 192 to facilitate placement around the upper bandage 60 and the tape 62. At least one bag of dessicant 102 is positioned in the interior chamber 85, preferably proximate the generally rectangular cutout 92 A second bag of dessicant 104 may also be included in the interior chamber 85 near the end of the sleeve 82. The dessicant is designed to absorb water vapor and water to keep the upper protective bandage 60 and the lower protective bandage 64 dry.

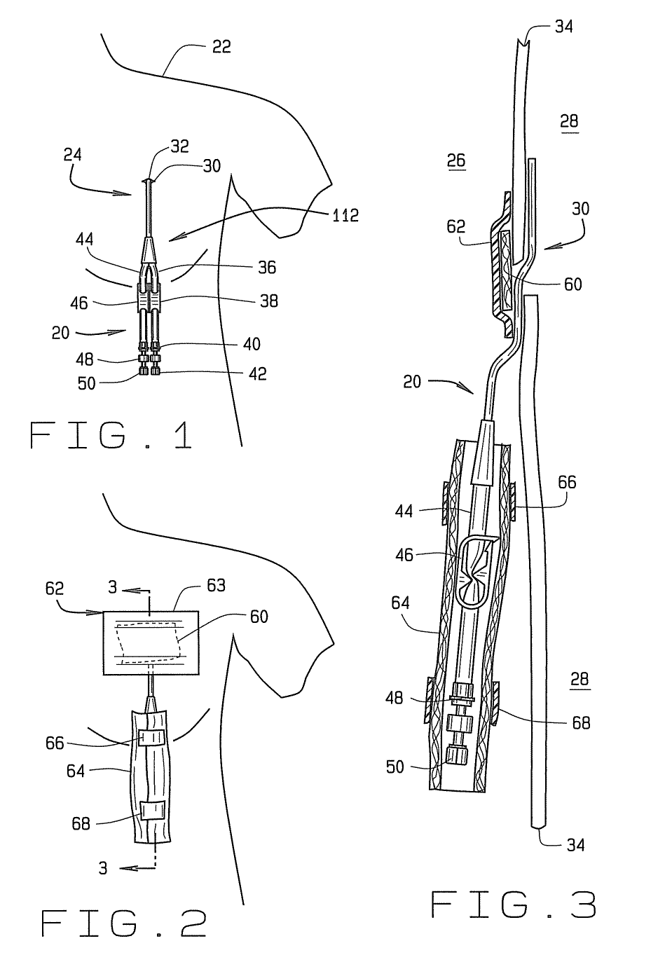

FIG. 7 is a section view of shower guard 80 surrounding the renal access catheter 20 which passes from the exterior of the patient 26 through the catheter entrance site 30 into the chest cavity 28 as is well known to those skilled in the art. As previously indicated in FIGS. 2 and 3, an upper protective bandage 60 is typically held in place by tape 62 over the catheter entrance site 30 on the exterior 26 of the patient 22, lower protective bandage 64 held in place by tape 66 and 68 will often surround the exposed portion 112 of the renal access catheter 20.

FIG. 8 is a top plan view of a portion of the shower guard showing the generally rectangular cutout 92 fitted over the tape 62 and upper protective bandage 60. The shape of the generally rectangular cutout 92 is sized and arranged to fit over the generally rectangular tape 62 and upper bandage 60. The sleeve 82 hangs down to surround the catheter 20.

To install the shower guard 80, backing paper, not shown is removed from the posterior side 90 of the wafer 86 exposing the adhesive 94. The generally rectangular cutout 92 is then placed around the tape 62 leaving a surrounding margin 110 of exposed skin between the edge of the generally rectangular cutout 92 and the perimeter 63 of the tape 62, as better seen in FIG. 8.

The patient then takes a shower and after drying off, removes the shower guard 80. Because there is a surrounding margin 110 between the perimeter 63 of the tape 62 and the cutout 92, the upper protective bandage 60 and tape 62 are not disturbed by removal of the shower guard 80.

1. A disposable, single use guard for a patient having a catheter, an external portion extending through the skin of the patient at a catheter entrance site, the shower guard comprising:
a sleeve formed from waterproof material defining a chamber shaped to receive the external portion of the catheter and surround a protective bandaging at the catheter entrance site;
a wafer connected to a portion of the sleeve, one side of the wafer having adhesive to removably adhere the guard to the skin of the patient proximate the catheter entrance site;
a generally rectangular central cutout in the wafer and the sleeve sized to surround the catheter entrance site and be sufficiently large enough to leave a margin between an edge of the cutout and the protective bandaging at the catheter entrance site to leave the protective bandaging at the catheter entrance site undisturbed when the guard is removed from the patient; and
a least one package of desiccant positioned inside the chamber of the sleeve.

2. The apparatus of claim 1 further including a first package of desiccant positioned inside the chamber of the sleeve proximate the catheter entrance site and a second package of desiccant positioned inside the chamber of the sleeve proximate a terminus of the catheter.

3. The apparatus of claim 1 wherein the sleeve is formed from transparent material.

4. The apparatus of claim 1 further including a colored border surrounding at least a portion of the generally rectangular central cutout in the wafer.

5. The apparatus of claim 1 further including a colored border surrounding the generally rectangular central cutout in the sleeve.

6. A disposable, single use guard for a catheter having an internal portion implanted beneath the skin of a patient and an external portion passing through a catheter entrance site, the internal portion of the catheter being in fluid communication with the internal portion, the guard comprising:
a sleeve formed from waterproof material defining a chamber shaped to receive and enclose the external portion of the catheter and protective gauze;
a two-sided wafer having a generally rectangular central cutout sized to surround a generally rectangular piece of tape and gauze protecting the catheter entrance site, one side of the wafer connected to the sleeve and the other side of the wafer having adhesive, to removably adhere the guard to the skin of the patient; and
at least one bag of desiccant secured inside the chamber.

7. The apparatus of claim 6 wherein the sleeve is formed from transparent material.

8. The apparatus of claim 6 further including a colored border surrounding at least a portion of the generally rectangular central cutout in the wafer.

9. The apparatus of claim 6 further including a colored border surrounding the generally rectangular central cutout in the sleeve.

10. A disposable, single use shower guard for a renal dialysis patient having an access catheter, an external portion of the access catheter extending through the skin of the patient at a catheter entrance site which is covered by a generally rectangular protective bandage, the shower guard comprising:
a sleeve formed from waterproof material defining a chamber shaped to receive and surround the external portion of the access catheter, a protective bandage surrounding at least some of the external portion of the access catheter and the generally rectangular protective bandage covering the catheter entrance site;
a two sided wafer connected to the sleeve, at least a portion of one side of the wafer opposite the sleeve having adhesive, to removably adhere the shower guard to the skin of the dialysis patient proximate the catheter entrance site;
a generally rectangular central cutout in the two sided wafer and the sleeve sized to surround the generally rectangular protective bandage at the catheter entrance site and be sufficiently large to leave a margin between the cutout and the generally rectangular protective bandage over the catheter entrance site; and
at least one package of desiccant positioned inside the chamber of the sleeve.

11. The apparatus of claim 10 wherein the sleeve is formed from transparent material

12. The apparatus of claim 10 further including a colored border surrounding at least a portion of the generally rectangular central cutout in the wafer.

13. The apparatus of claim 10 further including a colored border surrounding the generally rectangular central cutout in the sleeve.
